(19) United States
(12) Patent Application Publication
(10) Pub. No.: US 2023/0301586 A1
(43) Pub. Date: Sep. 28, 2023

(54) SYSTEM AND METHOD FOR CHARACTERIZING, DETECTING AND MONITORING SLEEP DISTURBANCES AND INSOMNIA SYMPTOMS

(71) Applicant: Feel Therapeutics Inc., San Francisco, CA (US)

(72) Inventors: Georgios Eleftheriou, Santa Clara, CA (US); Panagiotis Fatouros, Athens (GR); Charalampos Tsirmpas, Athens (GR)

(21) Appl. No.: 18/126,100

(22) Filed: Mar. 24, 2023

Related U.S. Application Data
(60) Provisional application No. 63/323,750, filed on Mar. 25, 2022.

(51) Int. Cl. A61B 5/00; G16H 50/50; A61B 5/0205

(52) U.S. Cl. CPC A61B 5/4809; G16H 50/50; A61B 5/0205; A61B 5/4815

(57) ABSTRACT

One variation of a method includes: accessing a first timeseries of biosignal data collected by a wearable device worn by a user during a first time period; deriving a first insomnia profile, representative of a set of health indicators exhibited by the user during the first time period, based on the first timeseries of biosignal data; and selecting a treatment pathway for implementation by the user based on the first insomnia profile. The method further includes: accessing a second timeseries of biosignal data collected for the user by the wearable device during a second time period; deriving a second insomnia profile, representative of the set of health indicators exhibited by the user during the second time period, based on the second timeseries of biosignal data; and characterizing effectiveness of the treatment pathway based on a difference between the first insomnia profile and the second insomnia profile.

### CROSS-REFERENCE TO RELATED APPLICATIONS

This application claims the benefit of U.S. Provisional Application No. 63/323,750, filed on 25 Mar. 2022, which is incorporated in its entirety by this reference.

### TECHNICAL FIELD

This invention relates generally to the field of biosensors and more specifically to a new and useful method for characterizing and monitoring sleep disturbances and insomnia symptoms in the field of biosensors.

### BRIEF DESCRIPTION OF THE FIGURES

FIG. 1 is a flowchart representation of a method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

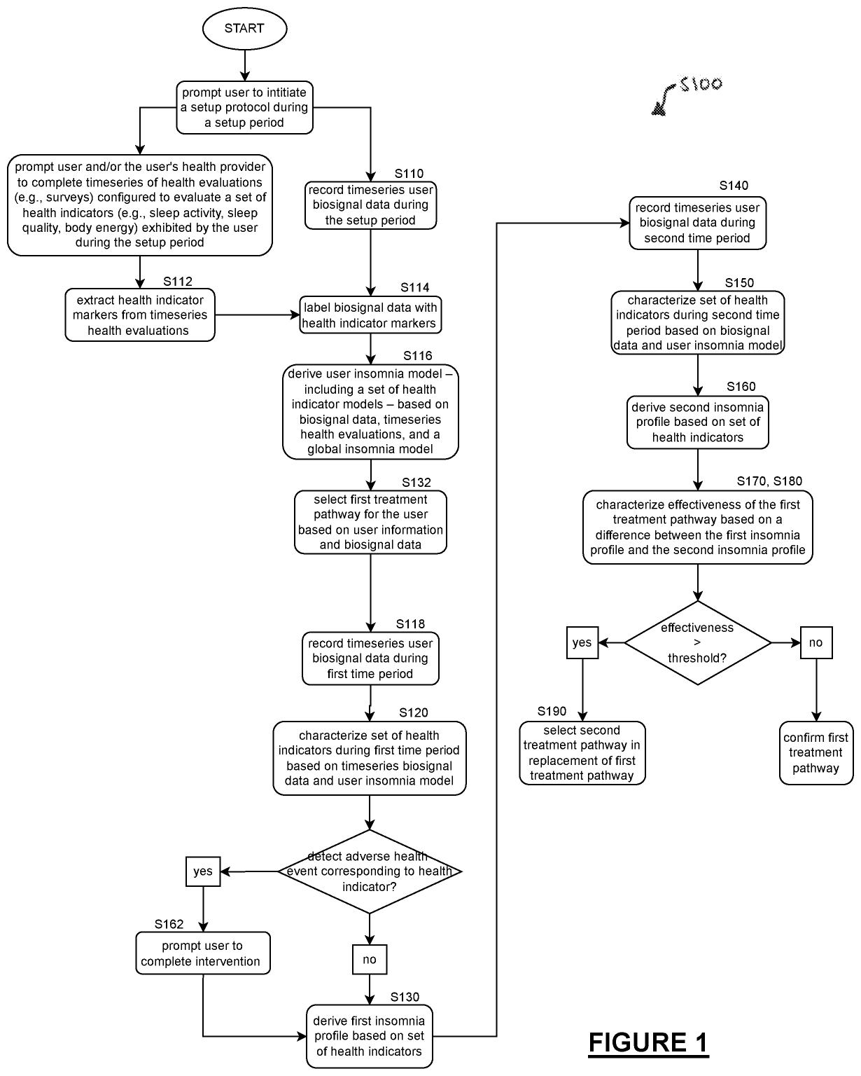

As shown in FIG. 1, a method S100 includes: during a first time period, accessing a first timeseries of biosignal data collected via a set of sensors integrated into a wearable device worn by the user during the first time period in Block S118; characterizing a set of health indicators exhibited by the user during the first time period based on the first timeseries of biosignal data in Block S120; deriving a first insomnia profile—representative of the set of health indicators during the first time period—for the user in Block S130; selecting a first treatment pathway for the user based on the first insomnia profile in Block S132; during a second time period, accessing a second timeseries of biosignal data collected via the set of sensors integrated into the wearable device worn by the user during the second time period in Block S140; characterizing the set of health indicators exhibited by the user during the second time period based on the second timeseries of biosignal data in Block S150; deriving a second insomnia profile—representative of the set of health indicators during the second time period—for the user in Block S160; characterizing a difference between the first insomnia profile and the second insomnia profile in Block Silo; characterizing effectiveness of the first treatment pathway based on the difference in Block S180; and in response to characterizing effectiveness of the first treatment pathway below a threshold effectiveness, selecting a second treatment pathway in replacement of the first treatment pathway in Block S190.

One variation of the method S100 includes, during a first time period: accessing a first timeseries of biosignal data collected via a set of sensors integrated into a wearable device worn by the user during the first time period in Block Silo; accessing a first timeseries of indicator markers extracted from a series of health evaluations for the user and including a first indicator marker for a first instance of a first adverse health state exhibited by the user during the first time period in Block S112; labeling the first timeseries of biosignal data according to the first timeseries of indicator markers to generate a first indicator-labeled timeseries of biosignal data in Block S114; deriving an insomnia model linking biosignals to indicator markers for the user based on the first indicator-labeled timeseries of biosignal data in Block S116; during a second time period succeeding the first time period, recording a second timeseries of biosignal data via the set of sensors integrated into the wearable device worn by the user in Block S150; detecting a second instance of the first adverse health state exhibited by the user based on the second timeseries of biosignal data and the insomnia model in Block S160; and, in response to detecting the second instance of the first adverse health state, selecting a first intervention, in a set of interventions, matched to the first adverse health state of the user in Block S162.

### 2. Applications

Generally, Blocks of the method S100 can be executed by a companion application executing on a mobile device in cooperation with a wearable device worn by a user and/or by a remote computer system (hereinafter the “system”) to: calibrate an insomnia model that links physiological biosignal data (e.g., heart-rate, heart-rate variability, skin temperature, sweat gland activity, electrodermal activity, etc.) recorded for the user to a set of health indicators (e.g., energy level, sleep quality, mental resilience, mood) exhibited by the user and related to insomnia; track and monitor changes in these health indicators exhibited by this user over time; detect adverse health events—such as a low energy level event, a low sleep event, and/or a negative mood event (e.g., a depressed mood event)—associated with adverse changes to the set of health indicators; suggest interventions to the user configured to mitigate detected adverse health events (or states) in near real-time; and evaluate effectiveness of various treatment pathways recommended to the user in managing the set of health indicators and/or alleviating adverse health events that may be caused by insomnia.

For example, the system can onboard a user seeking a better understanding of her insomnia and/or better coping strategies for managing symptoms of insomnia. In particular, the system can initially prompt the user to execute a series of health evaluations configured to evaluate the set of health indicators exhibited by the user over a particular test period. During execution of these health evaluations, the wearable device can record a timeseries of physiological biosignal data of the user via a suite of integrated sensors integrated into the wearable device. The wearable device can then offload the timeseries of physiological biosignal data to the remote computer system—such as via the mobile device in real-time or in intermittent data packets—and the remote computer system can then: extract a timeseries of health indicator levels—such as a timeseries of energy levels, a timeseries of sleep qualities, a timeseries of moods—exhibited by the user during the test period based on results of the series of health evaluations; synchronize these timeseries of health indicator levels with the timeseries of physiological biosignal data; and implement regression, machine learning, deep learning, and/or other techniques to derive links or correlations between these physiological biosignals and the set of health indicators exhibited by the user.

The system can implement similar techniques to derive correlations between biosignal data and additional health indicators (e.g., energy level, Circadian Rhythm, mental resilience, sleep quality, mood) exhibited by the user and compile each of these correlations into a user-specific insomnia model configured to predict magnitudes and/or changes to these health indicators exhibited by the user over time. Thus, for each health indicator, in the set of health indicators, the system can: learn a set of biomarkers (or “insomnia biomarkers”) indicative of a particular health state (e.g., a high or low body energy level, high or low sleep quality, high or low mental resilience)—corresponding to a particular health indicator(s)—when detected in the biosignal data for this user: and compile these insomnia biomarkers into the user-specific insomnia model.

The system can continue monitoring health states of the user over time to (regularly, continuously) refine the user-specific insomnia model and implement this model to identify intervention need and to serve intervention activities to the user in near real-time, thus enabling the user to monitor and mitigate instances of adverse health states or events caused by insomnia. For example, the system can periodically prompt the user to complete insomnia evaluations—while recording biosignal data of the user—configured to monitor the user's progression; and then confirm, refine, or reject the user-specific insomnia model based on results of these subsequent insomnia evaluations. Further, by learning a set of insomnia biomarker signals (e.g., insomnia biomarker expression) specific to the user and monitoring the user's biosignals on a regular basis (e.g., continuously, hourly, daily), the system can detect and alert (e.g., via the wearable device) the user of adverse health events as they occur in real or near-real time, enabling the user to extract insights into these events as well as react accordingly.

Additionally, the mobile device or companion application can load a particular intervention activity (e.g., a breathing activity, a journaling activity, a light exercise, a short resting period) matched to the health state of the user; and prompt the user to complete the particular intervention activity via the mobile device when the wearable device detects an instance of the adverse health state. By prompting the user (or “intervening”) during an adverse health event, the system can enable the user: to focus on mitigating the adverse health event by performing an intervention activity configured to improve a particular health indicator (e.g., energy level, sleep quality) associated with the adverse health event; and to isolate and record a circumstance that triggered the adverse health event.

Therefore, the system can leverage biosignal data of the user—in combination with evaluations performed by the user and/or the user's therapist—to: characterize an insomnia profile for the user based on a set of health indicators associated with the user's insomnia; detect instances of adverse health states and notify the user in near-real time; and suggest treatment pathways including intervention techniques matched to the user for mitigating the set of health indicators and therefore mitigating the user's insomnia.

The system is described below as executing Blocks of the method S100 to: derive correlations between biosignal data and health indicators (e.g., sleep quality, body energy, circadian rhythm, mental resilience, mood, stress index) exhibited by the user and compile each of these correlations into an insomnia model; and to characterize health states of the user—indicative of magnitudes and/or changes in these health indicators—based on the biosignal data and the insomnia model to monitor insomnia and/or symptoms of insomnia (e.g., chronic insomnia and/or acute insomnia) exhibited by the user. However, the system can implement similar methods and techniques to derive a model for any other sleep disorder and/or sleep disturbances (e.g., acute and/or chronic sleep disturbances).

### 3. Wearable Device & Companion Application

Blocks of the method S100 can be executed by a companion application executing on a mobile device in cooperation with a wearable device worn by a user and a remote computer system (or “the system”). The wearable device can record a timeseries of physiological biosignal data for the user via a set of sensors integrated into the wearable device. The wearable device can offload the timeseries of physiological biosignal data to the mobile device—such as in real-time or in intermittent data packets—and the companion application can return this data to the remote computer system. Additionally and/or alternatively, the wearable device can be configured to locally implement models (e.g., an insomnia model) to derive insights related to insomnia based on the timeseries of physiological biosignal collected at the wearable device.

Generally, the wearable device can access a set of sensors (e.g., an electrodermal activity sensor (or “EDA” sensor), a heart rate or photoplethysmogram sensor (or “PPG” sensor), a skin temperature sensor or (“STE” sensor), an inertial measurement unit (hereinafter “IMU”), an ambient humidity sensor, an ambient temperature sensor, a set of microphones) and record biosignal data at each sensor at a series of time increments.

For example, the system can access: the electrodermal activity sensor to record the skin conductance of the user; the heart rate sensor to record the pulse of the user; the IMU to record the motion of the user; the skin temperature sensor to record the user's skin temperature; the ambient humidity sensor to record the relative humidity of the air around the user; and the ambient temperature sensor to record the relative heat of the air around the user.

In one implementation, the wearable device can sample biosignal data intermittently (e.g., once per five-second interval) to reduce power consumption and minimize data files. In another implementation, the wearable device can selectively choose times to record biosignal data continuously instead of intermittently (e.g., if the system detects the user is trending toward an instance of an adverse health state, such as a low energy state). After recording the biosignal data at the wearable device, the wearable device can transmit the biosignal data to the mobile device for storage in the user's profile, such as locally on the mobile device and/or remotely in a remote database.

The method S100 is described as executed by and/or in conjunction with a wearable device. However, the method S100 can be executed by and/or in conjunction with any sensor(s) configured to record physiological biosignal data of the user.

### 3.1 Calibration

During a calibration period, the system can: load a companion application onto a user's mobile device; wirelessly (or via wired connection) connect the mobile device to the wearable device; run evaluations to confirm the functionality and accuracy of the set of sensors (e.g., an electrodermal activity sensor, a heart rate sensor, a skin temperature sensor, an inertial measurement unit (or “IMU”), an ambient humidity sensor, and an ambient temperature sensor) integrated into the wearable device; and prompt the user to enter demographic data (e.g., age, sex, education level, income level, marital status, occupation, weight, etc.) to generate a user profile. Generally, the companion application can—upon loading onto the mobile device—prompt the user to enter demographic user data to predetermine expected ranges of biosignal data for the user (e.g., a higher average skin temperature for a male user, a lower average skin temperature for a user of below average weight, a higher average skin conductance for a user in a high stress job, etc.).

Similarly, the wearable device can record a baseline resting heart rate, a baseline skin conductance, and a baseline level of activity for the user, and store all the baseline data locally on the wearable device or on the remote computer system as part of a user profile.

Furthermore, the wearable device can: access the set of sensors integrated into the wearable device worn by the user to acquire a first set of physiological biosignal data; and transmit the first set of physiological biosignal data to the mobile device. The companion application—executing on the user's mobile device—can then validate the first set of physiological biosignal data with generic baseline physiological biosignal data from a generic user profile (e.g., a profile defining a range of standard resting heart rates for a generic user, a range of normal skin temperatures, etc.), or from additional sources (e.g. confirming the ambient humidity recorded by the wearable device with the ambient humidity recorded by a third-party weather service, the time of day with the internal clock on the mobile device, etc.). For example, the wearable device can: record a particular physiological biosignal for the user (e.g., a resting heart rate); access a generic user profile including an acceptable (or expected) range for the particular biosignal (e.g., a resting heart rate between 60-100 beats per minute or “bpm”); and—if the biosignal data is within an acceptable range (e.g., 65 bpm)—store user biosignal data as a normal baseline for the user. Conversely, if the physiological biosignal data is not within the acceptable range (e.g., a resting heart rate of 40 bpm), the system can run diagnostics on the sensor or prompt the user to confirm the wearable device is on (or properly installed). The system can also prompt the user to override data that is out of the acceptable range (e.g., a marathon runner with a resting heart rate of 40 bpm can manually validate her resting heart rate.)

In another implementation, the companion application can: prompt the user to engage in a series of activities (e.g., sitting, walking, holding her breath, etc.); record a series of biosignal data via the set of sensors integrated into the wearable device; label the biosignal data with the associated activity; and store the labeled biosignal data in a user profile to enable the system to eliminate false positives triggered by normal activities.

### 3.2 Data Calibration: Environmental Controls

In one implementation, the wearable device records physiological biosignal data of the user (e.g., skin moisture, skin temperature, and heart rate variability) while concurrently recording ambient environmental data (e.g., humidity, ambient temperature) and other related data (e.g., the motion of the user or motion of the user's mode of transportation). For example, the wearable device can: access the electrodermal activity sensor to detect the user's current skin moisture data; identify that the user's current skin moisture data is above a normal threshold; access the ambient humidity sensor to detect an ambient humidity level; identify that the ambient humidity sensor is above the normal threshold; identify that the ambient humidity level is affecting the skin moisture data; and calculate a real skin moisture level based on the ambient humidity level. Therefore, the system can identify environmental situations that can affect the biosignal data of the user (e.g., washing her hands, running, etc.).

### 4. Insomnia Model

In one implementation, the system can prompt the user to schedule and/or execute a series of health evaluations configured to evaluate a set of health indicators (e.g., sleep quality, circadian rhythm, mental resilience, mood) exhibited by the user (e.g., at a particular time, over a particular time period) to derive a user-specific insomnia model for the user. Additionally and/or alternatively, in this implementation, the system can prompt the user and/or a health provider (e.g., a therapist, a pharmacist, a primary care provider, a health coach) associated with the user to manually provide feedback regarding health indicators exhibited by the user during recording of the timeseries of biosignal data.

In particular, the system can leverage results of user health evaluations to: identify instances of adverse health states—associated with the set of health indicators—exhibited by the user; synchronize the timeseries of biosignal data and instances of each adverse health state; and implement regression, machine learning, deep learning, and/or other techniques to derive links or correlations between these physiological biosignals and these adverse health states; and compile these correlations into a user-specific insomnia model—linking physiological biosignals with instances of various health states (e.g., high and/or low sleep quality, high and/or low body energy) exhibited by the user—such as by compiling these correlations into a new insomnia model for the user or by calibrating an existing generic insomnia model based on these correlations.

In this implementation, the system can: access a series of results of user health evaluations (e.g., a series of surveys submitted by the user and/or the user's therapist regarding presence of insomnia symptoms, a sleep study); and transform the series of results into timestamped instances (and magnitudes) of an adverse health state—such as a low energy level state, a low sleep quality state, or an adverse mood state—exhibited by the user while completing these health evaluations (e.g., based on a correlation between results of these health evaluations and adverse health states). The system can then: access the timeseries of biosignal data recorded during execution of the health evaluations; synchronize the timeseries of biosignal data and timestamped instances of the adverse health state; and implement regression, machine learning, deep learning, and/or other techniques to derive correlations between biosignal data and instances of the adverse health state for the user. The system can then store these correlations in a user-specific insomnia model generated for the user.

For example, the system can prompt the user to complete a survey—including sleep-related questions such as “How would you rate your sleep quality last night?” and/or “How well rested did you feel when you woke up this morning?”—configured to evaluate a sleep quality exhibited by the user during a particular sleep period. During the sleep period, the system can collect and record a timeseries of biosignal data via the set of sensors integrated into the wearable device worn by the user. The system can then: leverage results of the survey and the timeseries of biosignal data to characterize the sleep quality (e.g., a score between 0 and 100, a percentile compared to an average population) of the user during the sleep period based on historical biosignal data of other users diagnosed with insomnia; and link the sleep quality characterized for this sleep period to the timeseries of biosignal data collected during the sleep period. The system can then repeat this process—such as over multiple sleep periods—derive a set of sleep quality biomarkers (e.g., a particular heart rate range, a particular body temperature range) detectable in timeseries of biosignal data indicative of the sleep quality of the user.

The system can similarly extract biomarkers corresponding to other health indicators—such as energy level, mental resilience, or mood—and store these indicator biomarkers in the user-specific insomnia model. The system can then leverage this user-specific insomnia model in combination with physiological biosignal data to characterize health indicators exhibited by the user over time and/or delineate discrete adverse health events (e.g., low sleep quality, low energy level) associated with these health indicators.

Alternatively, in another implementation, the system can implement an existing generic insomnia model (e.g., a global insomnia model) configured to predict instances of health states and/or characterize the set of health indicators for users diagnosed with and/or exhibiting symptoms of insomnia.

### 4.1 User Insomnia Profile

The system can discern between different insomnia types or different health indicators associated with insomnia and exhibited by the user based on results of the series of user health evaluations, the timeseries of biosignal data, and/or a global or user-specific insomnia model. For example, the system can leverage magnitudes and patterns observed for particular biosignals recorded for the user to characterize an intensity of a particular health indicator exhibited by the user based on the user-specific insomnia model and the timeseries of biosignal data. The system can then leverage these health indicators—characterized for this particular user—to derive an insomnia profile for the user representative of magnitudes and/or changes in the set of health indicators during a particular time period.

For example, the system can: access a first timeseries of biosignal data collected over a first time period; characterize a first indicator score for a first health indicator, in the set of health indicators, based on the user-specific insomnia model and the first timeseries of biosignal data; characterize a second indicator score for a second health indicator, in the set of health indicators, based on the user-specific insomnia model and the first timeseries of biosignal data; and characterize a third indicator score for a third health indicator, in the set of health indicators, based on the user-specific insomnia model and the first timeseries of biosignal data. The system can then derive an insomnia profile for the user during the first time period based on the first, second, and third indicator scores. The system can then update this insomnia profile for the user over time as the system collects additional biosignal data.

### 4.2 Treatment Pathway

Based on the timeseries of biosignal data recorded during health evaluations and/or recorded over time for the user, the system can select a treatment pathway configured to monitor and/or improve the set of health indicators—represented by the user's insomnia profile—associated with insomnia (e.g., energy level, sleep quality, circadian rhythm, mental resilience, mood). For example, the system can select a treatment pathway including: a recommendation to meet with a mental health provider (e.g., a licensed therapist, a psychiatrist, a health coach) at a particular frequency; scheduled health evaluations configured to evaluate changes in these health indicators exhibited by the user; intervention activities matched to the user and configured to mitigate adverse health states; a supplement recommendation (e.g., a type and/or dosage of an over-the-counter supplement); a medication recommendation (e.g., a type and/or dosage of a prescription drug); and/or prompts to the user at a particular frequency soliciting feedback from the user. In one implementation, the system can initially select a generic treatment pathway for the user based on the initial insomnia profile of the user and/or based on the initial characterization of each of these health indicators. Over time, the system can continue evaluating biosignal data to modify and/or refine the treatment pathway to converge on a user-specific treatment pathway.

### 5. Health Indicators

In one implementation, the system can track and/or characterize a set of health indicators—such as sleep activity, sleep quality, energy level, mood, mental resilience, stress index, psychomotor activity, and/or cognitive state—which may be affected by the user's insomnia. In particular, the system can characterize each health indicator, in the set of health indicators, exhibited by the user based on timeseries of biometric data recorded for the user at a particular time and/or during a particular time period and the insomnia model, such as including a set of health indicator models (e.g., a sleep quality model, a body energy model, a psychomotor activity model, an emotion and/or mood model) and/or a set of sleep activity models.

The system can leverage these health indicators to: identify a particular subset of health indicators most relevant to this particular user; derive an insomnia profile unique to this user based on the set of health indicators exhibited by the user over time; characterize effectiveness of the user's current treatment pathway in improving the set of health indicators; and detect instances of adverse health events related to these health indicators.

For example, the system can derive a first insomnia profile for the user during a first time period based on a first timeseries of biosignal data collected for the user and the insomnia model, the first insomnia profile including: a first timeseries of sleep quality levels; a first timeseries of energy levels; and a first timeseries of user moods. Alternatively, in another example, the first insomnia profile can include: an average sleep quality corresponding to the first time period; an average energy level corresponding to the first time period; and an average frequency of a particular mood during the first time period.

### 5.1 Sleep Activity & Sleep Quality

In one implementation, the system can leverage timeseries of biometric data collected for the user to interpret the sleep quality of the user. In particular, the system can: interpret a set of sleep metrics—indicative of sleep activity for the user—based on timeseries of biosignal data recorded for the user; and interpret a sleep quality (e.g., an average sleep quality) for the user—such as corresponding to a particular night, a particular week, and/or a particular month—based on the set of sleep metrics.

In this implementation, the system can leverage a timeseries of biosignal data recorded during a sleep period for the user—detected by the system, specified by the user, and/or predefined for the user—to derive a set of sleep metrics for the user during this sleep period, such as: a quantity of sleep interruptions; a duration of each sleep interruption; a total duration of the sleep period (e.g., corresponding to a duration that the user was in bed and/or attempting to sleep); a total asleep duration (e.g., an amount of time the user is asleep) during the sleep period; a total awake duration (e.g., an amount of time the user is awake) during the sleep period; a duration of a “fall-asleep” period; an average duration of a REM cycle; a quantity of REM cycles completed during the sleep period; etc. The system can then interpret a sleep quality for the user during the sleep period based on these sleep metrics extracted from the timeseries of biosignal data.

In the preceding implementation, in order to enable detection of user sleep activity, the system can be configured to delineate a set of biomarkers for each sleep metric, in the set of sleep metrics, detectable in timeseries of biosignal data, such as based on results of user health evaluations (as described above) and/or known correlations between physiological biomarkers and the set of sleep metrics; and compile these interpreted biomarkers and/or correlations into a sleep activity model for the user. For example, the system can interpret: a first set of biomarkers corresponding to the user lying in bed and/or attempting to sleep; a second set of biomarkers corresponding to the user falling asleep; a third set of biomarkers corresponding to the user waking up; and/or a fourth set of biomarkers corresponding to a REM cycle. The system can then: compile the first, second, third, and fourth set of biomarkers into a sleep activity model for this user; and update the user-specific insomnia model to include this sleep activity model.

In one example, the system can: access a timeseries of biosignal data via the set of sensors integrated into the wearable device worn by the user; at a first time, detect initiation of a sleep period based on the timeseries of biosignal data (e.g., based on detected changes in the user's heart rate, body temperature, stress index) and the insomnia model generated for the user; and, in response to detecting initiation of the sleep period, label the timeseries of biosignal data with an initial sleep marker at the first time. Then, at a second time (e.g., in the morning) succeeding the first time, the system can: detect termination of the sleep period (e.g., when the user wakes up) based on the timeseries of biosignal data; and, in response to detecting termination of the sleep period, label the timeseries of biosignal data with a final sleep marker at the second time. In this example, the system can leverage the timeseries of biosignal data (e.g., recorded during the sleep period) to delineate additional sleep markers—such as corresponding to breaks in sleep, REM cycles, stress levels, etc.—and append the timeseries of biosignal data with these additional sleep markers accordingly. The system can then leverage these sleep markers to derive a set of sleep metrics for the user during the sleep period.

In the preceding example, the system can then leverage the set of sleep metrics derived from the timeseries of biometric data to characterize a sleep quality for the user during the sleep period. In particular, in one example, the system can characterize the sleep quality of the user during the sleep period based on: a total duration of the sleep period; and a sleep duration corresponding to a total amount of time the user slept during the sleep period. Alternatively, in another example, the system can characterize the sleep quality of the user during the sleep period based on a ratio of the sleep duration to an awake duration corresponding to a total amount of time the user was awake during the sleep period.

Additionally and/or alternatively, in another implementation, the system can directly interpret a sleep quality for the user (e.g., for a particular sleep period, over a particular time period) based on the timeseries of biosignal data. For example, in response to expiration of a sleep period for the user, the system can: access a timeseries of biometric data collected for the user during the sleep period; access an insomnia model—including a set of derived correlations (e.g., a sleep quality model) linking sleep quality to biometric data exhibited by the user—generated for the user; and interpret a sleep quality of the user during the sleep period based on the timeseries of biometric data and the insomnia model.

### 5.2 Body Energy

In one implementation, the system can leverage timeseries of biometric data collected for the user to interpret body energy of the user at a particular time of day and/or over a particular time period. In particular, the system can characterize body energy of the user based on a set of energy metrics extracted from timeseries of biosignal data for the user.

In one implementation, the system can characterize body energy of the user—such as for a particular time period (e.g., a 24-hour period)—based on a set of energy metrics, such as: an activity level of the user during the particular time period (e.g., characterized by a set of activity metrics derived from biosignal data of the user and/or from feedback provided by the user); a sleep quality of the user during the particular time period (e.g., characterized by a set of sleep metrics derived from biosignal data of the user and/or from feedback provided by the user); a mood or moods (e.g., stressed, relaxed, happy, sad, irritated) exhibited by the user during the particular time period and interpreted from biosignal data of the user; a diet of the user during the particular time period; etc.

Additionally and/or alternatively, the system can prompt the user to provide feedback related to the user's body energy. The system can then leverage this information provided by the user to confirm, disconfirm, and/or modify the insomnia model—including a set of health indicator models (e.g., a body energy model, a mood model, a psychomotor activity model) and/or a set of sleep activity models—for this user.

### 5.3.1 Physical & Psychomotor Activity

In one implementation, the system can leverage timeseries of biometric data collected for the user to characterize psychomotor and/or physical activity of the user at a particular time and/or during a particular time period (e.g., an hour, a day, a week, a month). In particular, the system can characterize a psychometric and/or physical activity level of the user based on a set of activity metrics extracted from timeseries of biosignal data recorded for the user.

For example, the system can leverage a timeseries of biosignal data recorded during an awake period for the user—detected by the system, specified by the user, and/or predefined for the user—to derive a set of physical activity metrics for the user during this awake period, such as: a number of instances of high-intensity physical activity detected during the awake period; a number of instances of moderate-intensity physical activity detected during the awake period; a duration of instances of high-intensity and/or moderate-intensity physical activity; a number of instances of reduced physical activity (e.g., below average activity level) detected during the awake period; a duration of each instance of reduced physical activity; etc. The system can then interpret a physical activity level of the user during the awake period based on these activity metrics extracted from the timeseries of biosignal data. The system can similarly leverage timeseries of biosignal data to derive a set of psychomotor activity metrics—such as related to user reaction time (e.g., speed of physical body movements) and/or coordination (e.g., balance, concentration)—and interpret a psychometric activity level (e.g., elevated, normal, and/or reduced psychometric activity) accordingly.

The system can continue to delineate instances of reduced-, low-, moderate-, and/or high-intensity activity throughout the awake period—based on the timeseries of biosignal data and the insomnia model—and append the timeseries of biosignal data with activity markers accordingly. The system can then leverage these activity markers to characterize an activity level of the user during the awake period, during a particular time period within the awake period, and/or over multiple awake periods.

### 5.3.2 Diet

In one implementation, the system can track characteristics of the user's diet (e.g., daily diet) to identify patterns and/or triggers associated with the user's diet that worsen and/or improve other health indicators exhibited by the user.

For example, the system can prompt the user to periodically (e.g., at each meal, daily, pseudo randomly) confirm (e.g., manually confirm) instances of food consumption by the user. Then, in response to receiving confirmation of a first instance of the user eating a meal (or a first “meal instance”), the system can: access a timeseries of biometric data recorded during, before, and/or after the first meal instance; characterize a first insomnia score (e.g., corresponding to a magnitude of insomnia symptoms exhibited by the user before and/or leading up to eating her meal) for the user during a first time period—within the daily tracking period—immediately preceding the first time (e.g., during a i-hour window preceding the first time) based on a first subset of timeseries of biometric data, in the timeseries of biometric data, corresponding to the first time period; and characterize a second insomnia score (e.g., corresponding to a magnitude of insomnia symptoms exhibited by the user after eating her meal) for the user during a second time period—within the daily tracking period—succeeding the first time (e.g., during a i-hour window succeeding the first time) based on a second subset of timeseries of biometric data, in the timeseries of biometric data, corresponding to the second time period. The system can then: characterize a difference between the first insomnia score and the second insomnia score; and, in response to the difference exceeding a threshold difference, flag this first meal instance for further investigation.

For example, the system can: characterize the first meal instance as a positive trigger (e.g., a trigger that reduces an extent of insomnia symptoms) in response to the first insomnia score exceeding the second insomnia score; and characterize the first meal instance as a negative insomnia trigger (e.g., a trigger that increases an extent of insomnia symptoms) in response to the second insomnia score exceeding the first insomnia score. Additionally and/or alternatively, in this example, the system can: generate a marker in the timeseries of biometric data at the first time corresponding to the meal instance; link the first insomnia score, the second insomnia score, and the difference to the marker; and prompt a health professional associated with the user to investigate this marker.

### 5.4 Mood

In one implementation—as described in U.S. patent application Ser. No. 16/460,105, filed on 2 Jul. 2019, which is incorporated in its entirety by this reference—the system can track the user's emotions (or “moods”) throughout the day. In particular, the system can leverage physiological biosignal data of the user—collected over a period of time—to delineate specific emotions exhibited by the user during this period of time.

For example, during a setup period, the companion application can: prompt the user to recall a story associated with a target emotion (e.g., happy, sad, stressed, distressed, etc.); and capture a voice recording of the user orally reciting this story. During the user's recitation of this story, the wearable device can record a timeseries of physiological biosignal data of the user. The remote computer system can then: access the voice recording; extract timeseries of pitch, voice speed, voice volume, pure tone, and/or other characteristics of the user's voice from the voice recording; and transform these timeseries of pitch, voice speed, voice volume, pure tone, and/or other characteristics of the user's voice into timestamped instances (and magnitudes) of the target emotion exhibited by the user while reciting the story. The remote computer system can then: access the timeseries of physiological biosignal data of the user recorded during recitation of the story; synchronize these timeseries of physiological biosignal data and instances of the target emotion; and implement regression, machine learning, deep learning, and/or other techniques to derive links or correlations between these physiological biosignals and the target emotion for the user.

The companion application, the wearable device, and the remote computer system can repeat this process to derive correlations between physiological biosignal data and other target emotions, such as during a single (e.g., ten minute) setup process or during intermittent setup periods during the user's first day or week wearing the wearable device. The remote computer system can then compile these correlations between physiological biosignal data and target emotions into an emotion model unique to the user, such as by compiling these correlations into a new emotion model for the user or by calibrating an existing generic emotion model to align with these correlations.

The mobile device can then: load a local copy of this emotion model to the wearable device (e.g., via the mobile device); record timeseries physiological biosignal data of the user via its integrated biosensors; and locally interpret the user's emotions in (near) real-time based on these timeseries physiological biosignal data. The system can then leverage this emotion model to interpret emotions of the user at a particular time and/or during a particular time period. In particular, the system can: delineate instances of various emotions (e.g., happy, sad, depressed, stressed, relaxed) and/or interpret magnitudes of these emotions exhibited by the user—such as a stress index, a happiness index, and/or a sadness index—based on the timeseries of biosignal data; and append the timeseries of biosignal data with emotion markers accordingly. The system can then leverage these emotion markers to characterize the user's mood during a particular time period.

### 6. Daily Biosignal Tracking

The system can continue recording biosignal data for the user over time to further refine the user-specific insomnia model and update the user's insomnia profile accordingly. In one implementation, the system can monitor biosignal data for the user each day (e.g., for three days, for one week, for two weeks) to identify patterns and/or trends in the observed biosignal data over the course of the user's day. The system can then store these observed patterns and/or trends in the insomnia profile for the user.

For example, the system can record a timeseries of biosignal data at set intervals (e.g., once per minute, once every ten minutes, three times per hour) each day during a set time period (e.g., one week). Then, in response to detecting a first instance of an adverse energy level (e.g., a low energy state) between 12 PM and 5 PM each day, the system can store these instances of the adverse energy level in the insomnia profile associated with the user. The system can then: predict that the user may experience an instance of an adverse energy level each day between 12 PM and 5 PM based on the insomnia profile; and inform the user of this prediction, such that the user may better plan her day around these predicted instances of the adverse energy level and/or implement intervention strategies—as discussed below—to mitigate these adverse energy levels. The system can continue tracking biosignal data over time and refine these observed patterns and/or trends as the system acquires additional data and updates and/or modifies the insomnia profile as these patterns and/or trends change over time.

Similarly, the system can identify patterns related to other health indicators exhibited by the user throughout the day—such as an elevated energy level, a positive mood, and/or a reduced stress level—linked to baseline or improved energy level, mood, and/or stress level for the user. The system can then leverage these patterns to identify and signal to the user the best conditions (e.g., time of day, environment) for maximum productivity and/or minimum user interruption due to these health indicators. For example, the system can prompt the user to complete the most difficult tasks of the day during a first time period associated with an elevated energy level for the user and limit the quantity and/or difficulty of tasks performed during a second time period associated with reduced energy level for the user. The system can thus store this information (e.g., frequency, duration, times of day, patterns) regarding instances of worsened and/or improved health indicators in the insomnia profile for the user.

### 6.3 Intervention Exercises

In one implementation, the system can prompt the user to complete an intervention exercise in response to detecting an instance of an adverse health state (e.g., reduced energy level, reduced sleep quality, reduced mental resilience) for the user.

In this implementation, the system can: detect an instance of the adverse health state via the wearable device; via the user's mobile device, load an intervention protocol (e.g., a set of intervention activities geared to move past the adverse health state); and prompt the user to complete an intervention activity (e.g., a breathing exercise, a mood diary, cognitive and behavioral exercises, and activities which are tailored to the user and/or a particular health indicator, etc.). The system can suggest different intervention activities based on type (e.g., energy level, sleep quality), intensity, timing, and/or duration of adverse health events.

For example, the system can: access the set of sensors on the wearable device, detect an instance of an adverse health state (e.g., low energy level) corresponding to a particular health indicator; access the insomnia profile of the user to select a first intervention exercise—matched to the user and the adverse health state—based on the treatment pathway defined for the user; signal to the user by vibrating the wearable device; and prompt the user via the mobile device to begin a meditative activity or a resting period to help restore the user's energy and regulate the particular health indicator (e.g., energy level) to within a target indicator zone corresponding to the particular health indicator and the insomnia profile of the user.

### 7. User Feedback

In one implementation, the system can prompt the user to periodically confirm instances of adverse and/or positive health indicators to further refine the user-specific insomnia model and/or insomnia profile for the user.

In one example, the system can periodically (e.g., once every morning, once per week, pseudo-randomly) prompt the user to complete a survey regarding instances of health indicators recorded for the user. In particular, in this example, the system can: generate a survey including a set of questions related to the user's sleep and energy level, such as: “On a scale from 1 to 10, how well did you sleep last night?”, or “On a scale from 1 to 5, how rested do you feel this morning?”, or “Does your energy level feel higher this morning that yesterday morning?”; transmit the survey to the user (e.g., to a mobile device accessed by the user) in the morning (e.g., within an hour of the user waking up); and, in response to receiving results of the survey from the user (e.g., from the mobile device accessed by the user), store results of the survey with a timestamp corresponding to a date and time of receiving results of the survey from the user.

In another example, in response to detecting a severe (e.g., high intensity, long duration) instance of an adverse energy level state, the system can prompt the user to record a brief journal entry detailing the instance of the adverse energy level state including: whether the user felt high- or low-energy; whether the user felt tired; whether the user felt productive; whether the user performed an intervention activity before, during, and/or after the instance of the adverse energy level state; a list of activities performed by the user before, during, or after the instance of the adverse energy level state; a list of supplements ingested by the user before, during, or after the instance of the adverse energy level state; diet information (e.g., types of food eaten, timing of meals and/or snacks, an amount of food eaten) for a time period including the instance of the adverse energy level state; etc.

The system can then leverage this information entered by the user to: confirm or reject instances of adverse or positive health indicators; identify false-negative and/or false-positive instances of adverse or positive health indicators; update the insomnia profile of the user; update the treatment pathway selected for the user; and/or select intervention activities for the user better matched to the user's insomnia profile.

### 8. Third-Party Feedback

In one implementation, the companion application can prompt the user to share all or part of her user profile with a third-party user (e.g., the user's licensed therapist) running the companion application on a different device such that the system can update the other user with certain data about the user (e.g., adverse cognitive states and trends).

For example, the system can: track instances of a particular adverse health event (e.g., corresponding to energy level, sleep quality, mood) for a particular period of time (e.g., a week) based on recorded biosignal data and the user-specific insomnia model; prompt the user to share a list of instances of the adverse health event from the particular period of time with her licensed therapist; and—upon receiving instructions from the user to share a list of instances of the adverse health event—send the list of instances of the adverse health event to the licensed therapist's device running the companion application. The licensed therapist may discuss these instances of the adverse health event with the user to extract further details regarding severity of these adverse health events and/or to discern false-positive and false-negative adverse health events. The licensed therapist may then leverage these details to generate feedback (e.g., via labelled biosignal data) from this therapy session for uploading to the system. Based on this feedback from the user's therapist, the system can update the user-specific insomnia model for the user to more accurately detect adverse health events in the future.

Additionally and/or alternatively, in the preceding the example, the system can be configured to selectively transmit notifications to the licensed therapist (e.g., via native application) indicating detected instances of adverse health events, such as corresponding to severe (e.g., high magnitude and/or duration) adverse health events.

The system can enable the third-party user to implement and/or suggest therapeutic techniques (e.g., behavioral therapy, supplements, medication) matched to the insomnia profile of the user. For example, rather than initially implementing strategies for treating a generic insomnia diagnosis, the system can enable the user's therapist to suggest a: medication and/or therapeutic technique linked to the set of health indicators—represented by the user's insomnia profile—exhibited by the user; and at a dosage associated with an intensity of these symptoms specified in the user's insomnia profile.

### 9. Evaluating Effectiveness of Therapeutics

The system can continue monitoring biosignal data of the user over time to continue refining the insomnia profile for the user and suggesting updated treatment pathways, intervention activities, and general tools for mitigating adverse effects of the user's insomnia on various health indicators that are best matched to the insomnia profile of the user.

As the system collects additional biosignal data from the user—in combination with feedback from the user and/or the user's licensed therapist—the system can converge on a refined user-specific insomnia profile. For example, the system can: monitor the user's biosignals on a daily basis; serve the user a series of health evaluations over a period of time (e.g., one month, one year); collect feedback from the user's licensed therapist over the period of time; collect feedback from the user over the period of time; serve and/or inform the user of intervention activities for completion during detected adverse health states; etc.

Further, based on this information collected over time (e.g., biosignal data, feedback from the user and/or the user's licensed therapist, results of health evaluations), the system can monitor and/or track the user's progress in management of her insomnia and/or health indicators (e.g., energy level, sleep quality). The system can inform the user and/or the user's mental health provider of this progress and leverage this information to modify and/or select treatment pathways over time. For example, over a six month period, the system can identify an increase in overall energy level of the user, such as based on frequency of adverse energy level states detected from biosignal data, intensity of biosignal data during these adverse energy level states, feedback from the user and her therapist, and/or results of health evaluations. The system can therefore leverage this data to reaffirm the user's current treatment pathway. Alternatively, if the system identifies a reduction in the user's overall energy level, the system can select a second treatment pathway in replacement of the first treatment pathway and configured to increase the user's energy level.

### 9.3 Supplement & Medication Effectiveness

In one implementation, the system can characterize effectiveness of a particular supplement (e.g., a type and/or dosage of an over-the-counter supplement) and/or of a particular medication (e.g., a type and/or dosage of a prescribed drug) in treating a sleep disorder (e.g., insomnia, sleep disturbances) and/or alleviating symptoms (e.g., decreased energy level, decreased sleep quality, depressive moods) of this sleep disorder.

In particular, the system can prompt the user and/or a third-party user (e.g., the user's therapist or health provider) associated with the user to provide information related to the user's medical treatment, such as: a list of supplements (e.g., over-the-counter supplements) and/or medications (e.g., prescription drugs) taken by the user; a dosage of each supplement and/or medication recommended for the user; a schedule for each supplement and/or medication, such as a frequency and/or particular time period recommended for the user to ingest or apply the supplement and/or medication; etc. The system can then: track a timeseries of biosignal data for the user; leverage the timeseries of biosignal data to interpret timeseries of instances of adverse health states and/or positive health states; and characterize effectiveness of a current treatment pathway—including these supplements and/or medications—assigned to the user based on these timeseries of instances of adverse and/or positive indicators.

For example, during a setup period, the system can prompt a user (e.g., via a native application executing on the user's mobile device) to provide information regarding the user's current treatment pathway for alleviating insomnia and/or indicators of insomnia. The system can then record a first medication (e.g., an oral medication) specified by the user in a user profile associated with the user. Additionally and/or alternatively, in this example, the system can prompt the user's therapist (e.g., via a native application executing on the provider's mobile device) to provide and/or confirm the user's current treatment and/or the first medication.

Then, during a first time period of a target duration (e.g., one day, one week, one month) succeeding the setup period, the system can: access a first timeseries of biosignal data recorded for the user during the first time period; characterize a first energy level of the user during the first time period based on the first timeseries of biosignal data; and characterize effectiveness of the first medication based on the first energy level, such as based on whether the first energy level exceeds a target energy level (e.g., defined for the user) or falls within a target energy level range.

Then, during a second time period of the target duration and succeeding the first time period, the system can: access a second timeseries of biosignal data recorded for the user during the second time period; characterize a second energy level of the user during the second time period based on the second timeseries of biosignal data; characterize a difference between the first energy level and the second energy level; and characterize effectiveness of the first medication based on difference. Alternatively, in this example, the system can similarly characterize effectiveness of the first medication during the second time period based on the second energy level. Therefore, in this example, the system can track changes in the user's energy level over time based on timeseries of biosignal data collected for the user to evaluate effectiveness of the first medication in improving the user's energy level (e.g., in reducing an effect of insomnia on the user's energy level).

The system can similarly track changes in other health indicators (e.g., sleep quality, circadian rhythm, mental resilience, mood) exhibited by the user to characterize effectiveness of a medication, a dosage of a medication, and/or a medication schedule in reducing an effect of insomnia on these health indicators for the user.

Additionally, in this implementation, the system can: identify acute trends in biosignal data related to supplements taken by the user; and leverage these trends to extract insights related to acute effects of supplements taken by the user. The system can then provide guidance and/or suggestions to the user based on these detected acute effects.

For example, the system can: access a timeseries of biosignal data collected for the user by the wearable device worn by the user; each day, prompt the user to confirm ingestion of a particular supplement recommended for the user; and, in response to receiving confirmation of ingestion of the supplement at a first time, label the timeseries of biosignal data with a first supplement marker corresponding to ingestion of the supplement by the user. The system can then: characterize a first set of health indicators—such as a first energy level, a first sleep quality, a first mood, a first mental resilience—during a first time period succeeding the first time based on a first subset of the timeseries of biosignal data corresponding to the first time period; characterize a second set of health indicators—such as a second energy level, a second sleep quality, a second mood, a second mental resilience—during a second time period succeeding the first time based on a second subset of the timeseries of biosignal data corresponding to the second time period.

The system can then compare the first set of health indicators to the second set of health indicators to extract insights related to acute effects of the supplement on these health indicators exhibited by the user. Additionally and/or alternatively, the system can transmit this data—including a series of supplement markers recorded over time and/or a series of health indicators—to the user's therapist and/or health provider for further investigation and/or discussion with the user.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method comprising:
during a first time period:
accessing a first timeseries of biosignal data collected via a set of sensors integrated into a wearable device worn by a user during the first time period;
characterizing a set of health indicators exhibited by the user during the first time period based on the first timeseries of biosignal data;
deriving a first insomnia profile representative of the set of health indicators exhibited by the user during the first time period; and
selecting a first treatment pathway for the user based on the first insomnia profile; and
during a second time period succeeding the first time period:
accessing a second timeseries of biosignal data collected via the set of sensors integrated into the wearable device worn by the user during the second time period;
characterizing the set of health indicators exhibited by the user during the second time period based on the second timeseries of biosignal data;
deriving a second insomnia profile representative of the set of health indicators exhibited by the user during the second time period;
characterizing a difference between the first insomnia profile and the second insomnia profile;
characterizing effectiveness of the first treatment pathway based on the difference; and
in response to characterizing effectiveness of the first treatment pathway below a threshold effectiveness, selecting a second treatment pathway in replacement of the first treatment pathway.

2. The method of claim 1:
further comprising, during an initial time period preceding the first time period:
accessing an initial timeseries of biosignal data collected via the set of sensors integrated into the wearable device worn by the user during the initial time period;
accessing a timeseries of indicator markers derived from a series of health evaluations executed for the user and representative of the set of health indicators for the user during the initial time period;
labeling the initial timeseries of biosignal data according to the timeseries of indicator markers to generate a first indicator-labeled timeseries of biosignal data; and
deriving an insomnia model linking biosignal data to the set of health indicators for the user based on the first indicator-labeled timeseries of biosignal data;
wherein characterizing the set of health indicators exhibited by the user during the first time period based on the first timeseries of biosignal data comprises characterizing the set of health indicators exhibited by the user during the first time period based on the first timeseries of biosignal data and the insomnia model; and
wherein characterizing the set of health indicators exhibited by the user during the second time period based on the second timeseries of biosignal data comprises characterizing the set of health indicators exhibited by the user during the second time period based on the second timeseries of biosignal data and the insomnia model.
